(19) United States
(12) Patent Application Publication
(10) Pub. No.: US 2011/0289662 A1
(43) Pub. Date: Dec. 1, 2011

(54) GOGGLES WITH VISCOUS FOAM SEAL

(75) Inventor: Seth Neary, Burlington, VT (US)

(73) Assignee: Gordini U.S.A. Inc., Essex Junction, VT (US)

(21) Appl. No.: 12/788,738

(22) Filed: May 27, 2010

(51) Int. Cl. A61F 9/02

(52) U.S. Cl. 2/440

(57) ABSTRACT

Goggles comprising a protective lens, a frame member and a viscous foam layer comprising a viscous foam portion.

### FIELD OF THE INVENTION

The present invention relates to the field of goggles, including protective goggles such as for sports such as skiing and snowboarding, which conform to the user's face.

### BACKGROUND OF THE INVENTION

Protective goggles are well known and useful for protecting the user's eyes during work or activities such as sports, including skiing and snowboarding. Because users of protective goggles often wear the goggles for extended periods, and sometimes all day, comfort is a significant factor.

Additionally, many environments in which goggle are used require a secure fit to keep out the elements. For example, when skiing or snowboarding in a generally cold environment, it is necessary to keep wind out of the user's eyes and to create a controlled environment across the area of the user's eyes.

In keeping, there is needed a goggle that provides comfort, a sure fit creating a controlled environment and protection for the eyes of the user.

### SUMMARY OF THE INVENTION

The present invention is directed to goggles comprising a protective lens, a frame member and a viscous foam layer comprising a viscous foam layer. Preferably, the goggles further comprise a fleece covering on the viscous foam layer formed of a wicking material.

The viscous foam layer is preferably permanently attached to the frame member. Any suitable attachment can be used, including adhesives, heat bonding, or any known means or material to provide an attachment that will not release without destroying the material of the viscous foam layer.

Similarly, when the fleece covering is used, it is also preferred that the fleece material be permanently attached to material of the viscous foam layer and/or the frame member. As such, the fleece covering material can be attached by any known means, including adhesives, heat bonding, etc., such that the fleece covering cannot be removed without destroying the fleece covering and/or the material of the viscous foam layer.

Of course, the fleece covering need not be used if the material of the viscous foam layer has a combed finish that provides a comfortable surface for resting against the user's face. However, the fleece covering is a preferred embodiment since it is considered an improvement that provides wicking action to remove perspiration that may build up in the area of the goggles.

The viscous foam layer can be formed as a laminate of the viscous foam material with another material, such as a foamed rubber material. In such an embodiment, the viscous foam material can be placed in close relation to the frame, with the foamed rubber material toward the user's face, or with the foam rubber material in close relation to the frame with the viscous foam material toward the user's face. In any such variation, the use of a fleece covering is optional.

When a fleece covering is used, the fleece is preferably formed of a polyester, polyethylene or similar material that covers at least the area of the viscous foam layer that comes into contact with the user's skin. Preferably, however, the fleece covers the entire exposed surface of the viscous foam layer, from one side of the frame member to the other.

### BRIEF DESCRIPTION OF THE DRAWINGS

The present invention will be better understood when considered in view of the attached drawings, in which like reference characters indicate like parts. The drawings, however, are presented merely to illustrate the preferred embodiment of the invention without limiting the invention in any manner whatsoever.

FIG. 1 is a cross sectional view of a first embodiment of the goggles of the present invention through the upper contact area of the goggles on the user's face.

FIG. 2 is an exploded cross sectional view of the first embodiment of the goggles of the present invention through the upper contact area of the goggles on the user's face.

FIG. 3 is a cross sectional view of a second embodiment of the goggles of the present invention through the upper contact area of the goggles on the user's face.

FIG. 4 is an exploded cross sectional view of the second embodiment of the goggles of the present invention through the upper contact area of the goggles on the user's face.

FIG. 5 is a top view of the second embodiment of the goggles of the present invention.

FIG. 6 is a cross sectional view of the second embodiment of the goggles of the present invention through the middle of the goggles.

FIG. 7 is a cross sectional view of a third embodiment of the goggles of the present invention through the upper contact area of the goggles on the user's face.

FIG. 8 is an exploded cross sectional view of the third embodiment of the goggles of the present invention through the upper contact area of the goggles on the user's face.

### DETAILED DESCRIPTION OF THE PREFERRED EMBODIMENT

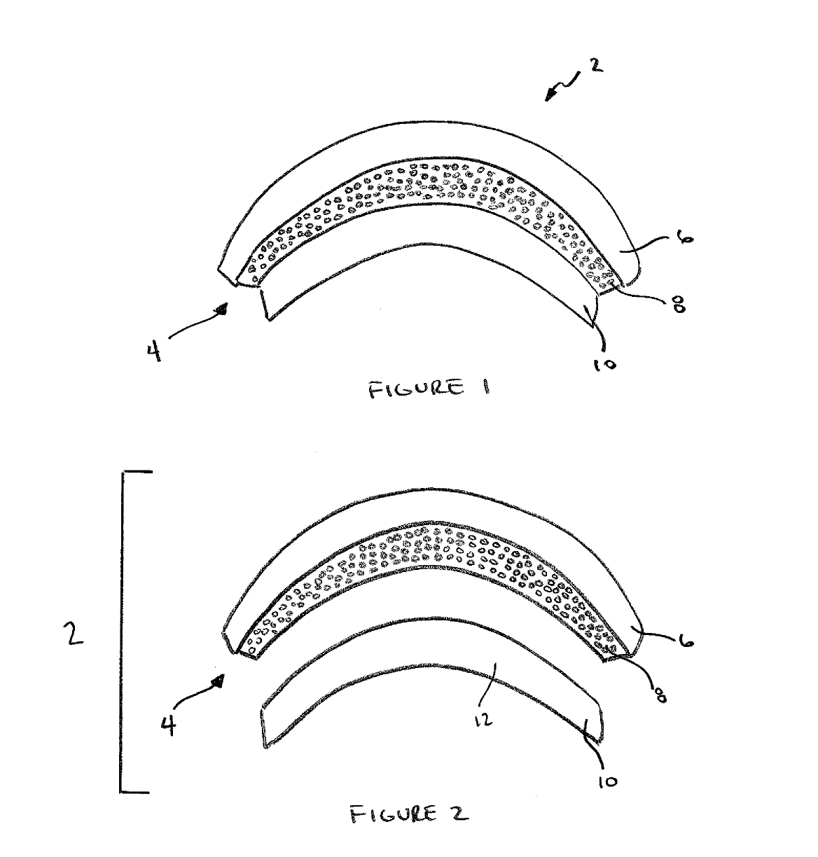

In the first embodiment, shown in the accompanying FIGS. 1-2, the goggles 2 of the present invention comprise a frame member 4, including a protective lens in a first frame portion 6 and a ventilation structure in a second frame portion 8, and a viscous foam layer 10. In the embodiment shown, the viscous foam layer 10 consists of a viscous foam portion 12.

In this embodiment, where the viscous foam layer 10 is formed entirely of a viscous foam portion 12, the viscous foam material of the viscous foam portion 12 rests directly against the user's skin. To ensure user comfort, at least the area of the viscous foam layer 10 that rests directly against the user's face has a combed surface that feels soft against the user's skin.

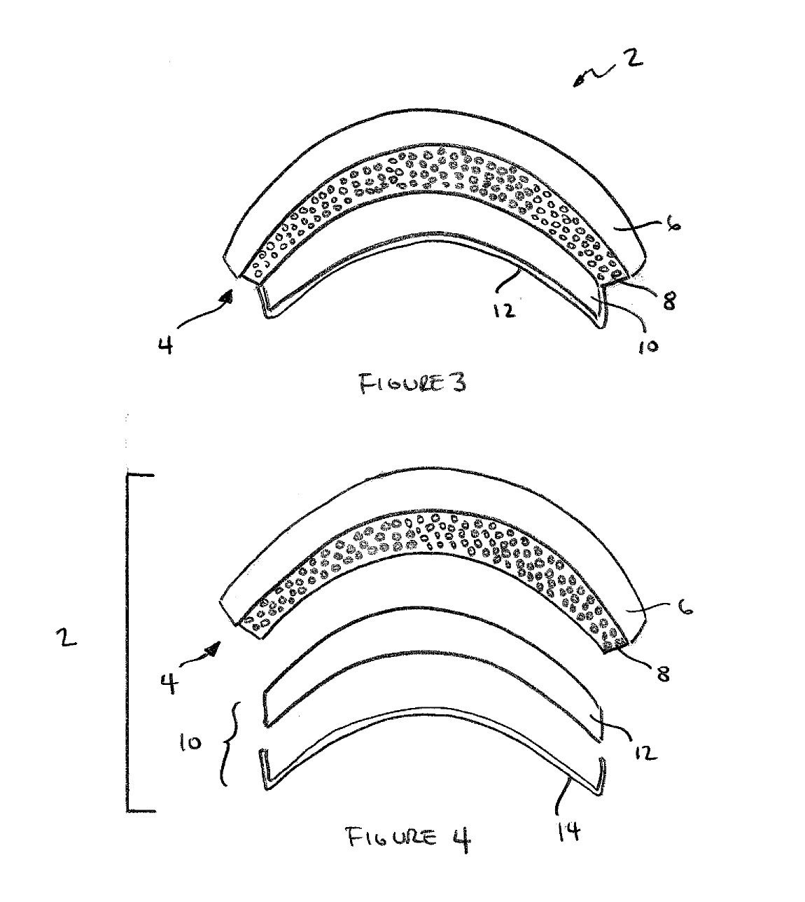

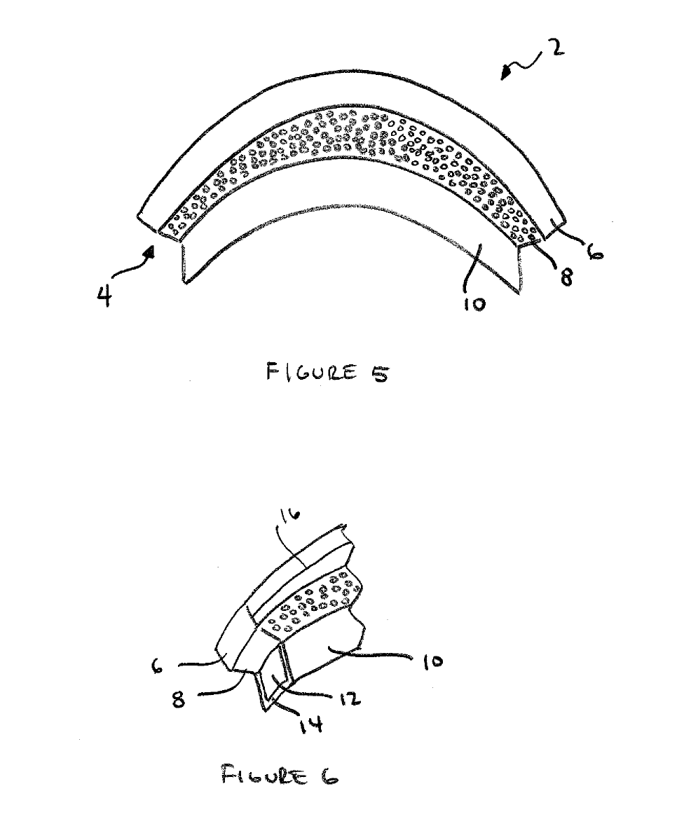

In the second embodiment, shown in FIGS. 3-6, the viscous foam layer 10 includes a viscous foam portion 12 and a fleece covering 14. The fleece covering 14 is preferably formed of a fleece piece material, and most preferably a polyester, polyethylene or similar material that provides wicking action for moving perspiration away from the user's skin. The fleece covering 14, when used, is formed at least in the area of the viscous foam layer 10 that comes into contact with the user's skin.

The preferred structure of this embodiment, shown in FIG. 6 (comprising a cross sectional view in the middle of the goggles 2 with the lens 16 illustrated), has the fleece covering 14 over the entire exposed surface of the viscous foam layer 10, from one side of the frame member 4 to the other.

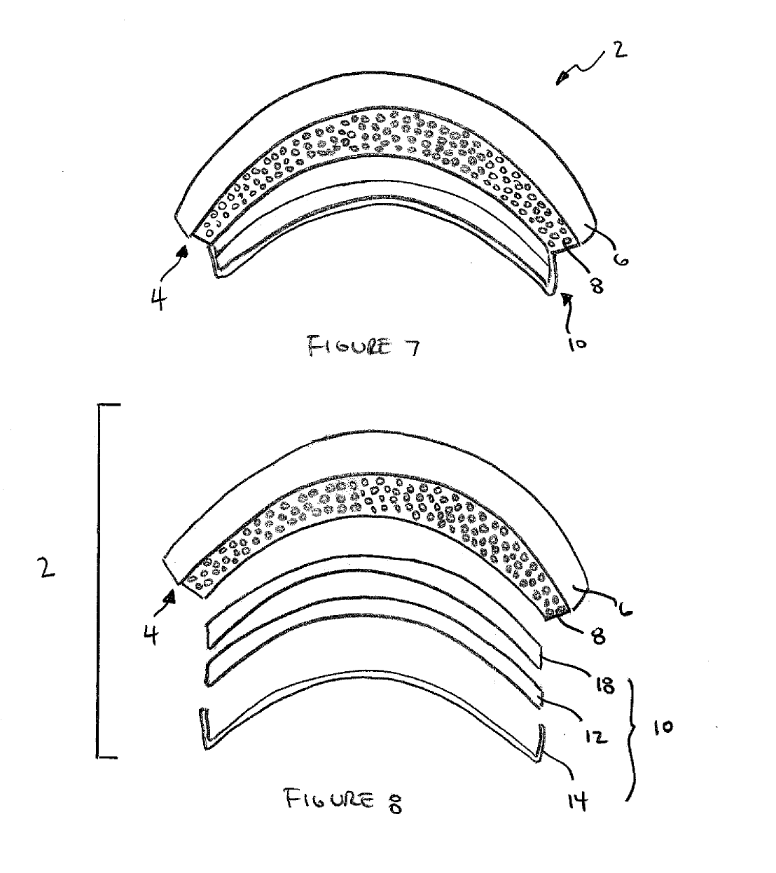

In the third embodiment shown in FIGS. 7 and 8, the viscous foam layer 10 is a laminate structure that includes a viscous foam portion 12, a secondary sublayer 18 and a fleece covering 14. Although this embodiment is shown with the secondary sublayer 10 between the frame member 4 and the viscous foam portion 12, it is understood that the viscous foam portion 12 may be formed between the frame member 4 and the secondary sublayer 18.

Additionally, it is preferred that when the viscous foam layer 10 includes a viscous foam portion 12 and a secondary sublayer 18, the viscous foam portion 12 comprises a stratum about the entire viscous foam layer 10.

In the preferred embodiment, the viscous foam material is a flexible polyurethane material having a density of from about 0.4 to about 1.0 PCF (pounds per cubic foot). Preferably, the viscous foam material is from about 0.35 to about 0.7 inches thick about the entire perimeter of the frame member that contacts the user's face.

The viscous foam layer 10 can be attached to the frame member 4 by any known means or method, such that viscous foam layer 10 is preferably permanently attached to the frame member. Suitable means and methods include, but are not limited to adhesives, heat bonding, or the like to provide an attachment that will not release without destroying the material of the viscous foam layer 10.

Similarly, when the fleece covering 14 and/or laminate structure including a secondary sublayer 18 is used, it is also preferred that the fleece covering 14 be permanently attached to viscous foam 12, secondary sublayer 18 and/or the frame member. As such, the fleece covering 14 can be attached by any known means or method, including but not limited to adhesives, heat bonding, etc., such that the fleece covering cannot be removed without destroying the fleece covering and/or the viscous foam layer 10.

The present invention provides that when the user places the goggles on his/her face, the viscous foam layer will conform to the user's face and provide a more comfortable environment. Although the use of such viscous foam material in climates that require ski or snowboard goggles has not been considered due to the fact that viscous foam is generally hard and uncomfortable in cold conditions, the present invention overcomes the concerns and surprisingly provides a comfortable environment for the user.

Variations, modifications and alterations to the preferred embodiment of the present invention described above will make themselves apparent to those skilled in the art. All such changes are intended to fall within the spirit and scope of the present invention, limited solely by the appended claims.

I claim:

1. Goggles comprising a protective lens, a frame member and a viscous foam layer comprising a viscous foam portion.

2. The goggles of claim 1 wherein the viscous foam layer further comprises a fleece covering over at least a portion thereof

3. The goggles of claim 2 wherein the fleece covering covers the portion of the viscous foam layer corresponding to the portion adapted to rest against the skin of a user when the goggles are being worn.

4. The goggles of claim 2 wherein the fleece covering covers the entire exposed surface of the viscous foam layer.

5. The goggles of claim 2 wherein the fleece covering is formed of a polyester or polyethylene material.

6. The goggles of claim 1 wherein the viscous foam layer is a laminated structure and the viscous foam portion comprises a stratum about the entire viscous foam layer.

7. The goggles of claim 6 where the viscous foam portion is the only sublayer of the viscous foam layer.

8. The goggles of claim 6 wherein the viscous foam layer includes a secondary sublayer.

9. The goggles of claim 6 wherein the viscous foam layer further comprises a fleece covering over at least a portion thereof

10. The goggles of claim 9 wherein the fleece covering covers the entire exposed surface of the viscous foam layer.

11. The goggles of claim 1 wherein the viscous foam portion is formed of a flexible polyurethane material.

12. The goggles of claim 11 wherein the flexible polyurethane material has a density of from about 0.4 to about 1.0 pounds per cubic foot.

13. The goggles of claim 1 wherein the viscous foam portion is a stratum about the entire viscous foam layer that is from about 0.35 to about 0.7 inches thick.

14. The goggles of claim 1 wherein the viscous foam layer is permanently attached to the frame member.

15. The goggles of claim 14 wherein the viscous foam layer is permanently attached to the frame member by an adhesive or by heat bonding.

16. The goggles of claim 2 wherein the fleece covering is permanently attached to the viscous foam layer.

17. The goggles of claim 16 wherein the fleece covering is permanently attached to the viscous foam layer by an adhesive or by heat bonding.
